(19) United States
(12) Patent Application Publication
(10) Pub. No.: US 2026/0360484 A1
(43) Pub. Date: Nov. 12, 2026

(54) INFORMATION PROCESSING SYSTEM AND INFORMATION PROCESSING METHOD

(71) Applicant: Hitachi High-Tech Corporation, Tokyo (JP)

(72) Inventors: Wataru TAKEUCHI, Tokyo (JP); Shunsuke HIDAKA, Tokyo (JP); Koichiro KIMOTSUKI, Tokyo (JP); Eichi WATABE, Tokyo (JP)

(73) Assignee: Hitachi High-Tech Corporation, Tokyo (JP)

(21) Appl. No.: 19/653,411

(22) Filed: Apr. 21, 2026

(30) Foreign Application Priority Data
May 12, 2025 (JP) 2025-080023

(51) Int. Cl. G16H 10/40; G16H 10/60

(52) U.S. Cl. CPC G16H 10/40; G16H 10/60

(57) ABSTRACT

An information processing system for an expert panel session includes: an input unit that receives inputs of a genetic test result and medical information of a patient who has undergone a genetic test, and a comment regarding clinical interpretation or treatment proposal for the genetic test result or the medical information of the patient from a participant of the expert panel session, and accumulates the inputs in a case database; an issue extraction unit that extracts an issue obtained by classifying and summarizing a plurality of the comments on the genetic test result and the medical information in the case database by a large language model and accumulates the extracted issue in an issue database.

### BACKGROUND OF THE INVENTION

### 1. Field of the Invention

The present invention relates to an information processing system and an information processing method for an expert panel including a molecular tumor board (MTB).

### 2. Description of the Related Art

In recent years, with the progress of information technology (IT), various systems that support judgment of experts have been developed. In particular, in a conference group called an expert panel, which consists of a plurality of experts who are well-versed in a specific field, there is a demand for more sophisticated judgment and a demand for supporting the experts to ensure accurate judgment.

For example, US 12,073,342 describes an invention of an informatics platform that provides an architecture for integrating information from a plurality of relevant patient information systems. The informatics platform includes a first tool that acquires first medical data of a patient from a database, a second tool that is linked to an application, and a third tool that has the following functions. The third tool has a function of displaying, on a graphical user interface, a region in which a third party application outside the system can be selected at the same time as the first medical data, and processing, when the third party application is selected, and the third party application acquires one or more clinical items related to the patient from the first medical data, the one or more clinical items to generate second medical data related to the patient. The third tool further has a function of receiving the second medical data from the third party application and a function of simultaneously displaying at least a part of the first medical data and the second medical data on the graphical user interface. The third tool provides a virtual conference interface via a graphical user interface, the virtual conference interface having a function of enabling a user to flag one or more items of at least a part of the first medical data and the second medical data for discussion at the conference.

### SUMMARY OF THE INVENTION

According to the invention described in US 12,073,342, information from the plurality of relevant patient information systems can be integrated. However, work of medical personnel related to the expert panel is not sufficient to merely integrate information from the plurality of patient information systems, and it is necessary to collect medical evidence necessary for a preparation stage for an expert panel session and generate a summary thereof, and to present issues of the expert panel session.

Therefore, an object of the present invention is to suitably support work of experts participating in an expert panel session.

In order to solve the above-described problems, an information processing system of the present invention is an information processing system for an expert panel session, the information processing system including: an input unit that receives inputs of a genetic test result and medical information of a patient who has undergone a genetic test, and a comment regarding clinical interpretation or treatment proposal for the genetic test result or the medical information of the patient from a participant of the expert panel session, and accumulates the inputs in a case database; an issue extraction unit that extracts an issue obtained by classifying and summarizing a plurality of the comments on the genetic test result and the medical information in the case database by a large language model and accumulates the extracted issue in an issue database; an issue candidate generation unit that extracts an issue related to a genetic test result or medical information of a patient different from the patient from the issue database by the large language model, and generates an issue candidate for the expert panel session for the patient based on the extracted issue; and a display control unit that displays the genetic test result and the medical information of the patient, and the issue candidate on a display unit.

That is, an information processing method of the present invention is an information processing method for an expert panel session, the information processing method including: receiving, by an input unit, inputs of a genetic test result and medical information of a patient who has undergone a genetic test, and a comment regarding clinical interpretation or treatment proposal for the genetic test result or the medical information of the patient from a participant of the expert panel session, and accumulating the inputs in a case database; extracting, by an issue extraction unit, an issue obtained by classifying and summarizing a plurality of the comments on the genetic test result and the medical information in the case database by a large language model and accumulating the extracted issue in an issue database; extracting, by an issue candidate generation unit, an issue related to a genetic test result or medical information of a patient different from the patient from the issue database by the large language model; generating, by the issue candidate generation unit, an issue candidate for the expert panel session for the patient based on the extracted issue; and displaying, by a display control unit, the genetic test result and the medical information of the patient, and the issue candidate on a display unit.

Other means will be described in the description of the preferred embodiments.

According to the present invention, it is possible to suitably support work of experts participating in an expert panel session.

### BRIEF DESCRIPTION OF THE DRAWINGS

FIG. 1 is a block diagram of an information processing system according to the present embodiment;

FIG. 2 is a configuration diagram of an information processing apparatus;

FIG. 3 is a diagram for describing original past case data;

FIG. 4 is a diagram for describing an issue database used for a retrieval-augmented generation (RAG) for issue generation;

FIG. 5 is a diagram for describing a medical information database used for the RAG for issue generation;

FIG. 6 is a diagram for describing case data of a support target;

FIG. 7 is a diagram for describing an issue generated by a large language model for generating an issue;

FIG. 8 is a diagram for describing a case database;

FIG. 9 is a flowchart of preliminary preparation processing for an expert panel session;

FIG. 10 is a flowchart of in-session processing of the expert panel session;

FIG. 11 is a flowchart of background processing;

FIG. 12 illustrates a comment input screen according to a related art;

FIG. 13 illustrates a comment input screen in preliminary preparation for the expert panel session; and

FIG. 14 illustrates a comment input screen during the expert panel session.

### DESCRIPTION OF THE PREFERRED EMBODIMENTS

Hereinafter, embodiments for carrying out the present invention will be described in detail with reference to the drawings.

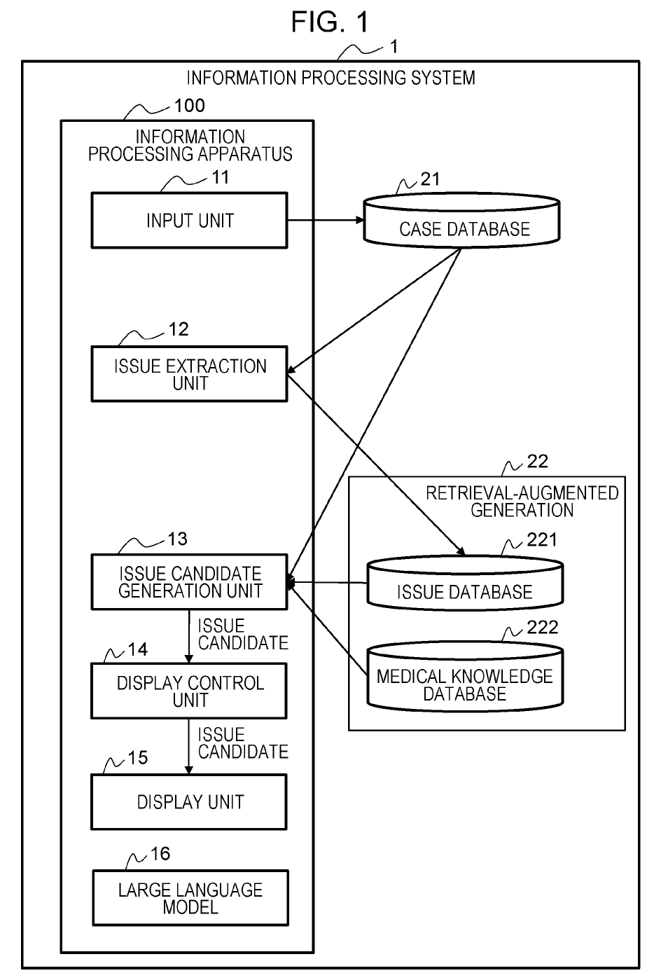

FIG. 1 is a block diagram of an information processing system 1 according to the present embodiment.

The information processing system 1 is a system that supports an expert panel, and includes an information processing apparatus 100. The information processing apparatus 100 includes, as functional units, an input unit 11, an issue extraction unit 12, an issue candidate generation unit 13, a display control unit 14, a display unit 15, and a large language model 16. Note that the large language model 16 may be a large language model which is called from outside the information processing apparatus 100, and is not limited thereto.

The information processing system 1 further includes a case database 21, an issue database 221, and a medical knowledge database 222. Retrieval-augmented generation 22 includes the issue database 221 and the medical knowledge database 222. Note that these various databases may be provided inside the information processing apparatus 100, and are not limited thereto.

The input unit 11 receives an input of a genetic test result and medical information of a patient who has undergone a genetic test, and accumulates the genetic test result and the medical information in the case database 21. Here, the medical information includes age, gender, disease name, case information, and treatment history.

The input unit 11 further receives, from a participant of an expert panel session, an input of a comment regarding clinical interpretation or treatment proposal for the genetic test result or the medical information of the patient and accumulates the comment in the case database 21.

The issue extraction unit 12 extracts an issue obtained by classifying and summarizing a plurality of the comments on the genetic test result and the medical information of the patient in the case database 21 using the large language model 16, and accumulates the extracted issue in the issue database 221. When the issue is created by classifying and summarizing the comments, the issue extraction unit 12 records the issue in the issue database 221 while dividing the issue into a plurality of elements including a keyword, a category, and a consideration item.

The issue candidate generation unit 13 receives the genetic test result and the medical information of the patient from the case database 21 by the large language model 16, extracts the issue related to the genetic test result or the medical information from the issue database 221, and generates an issue candidate for the expert panel session for the patient based on the extracted issue. The issue candidate generation unit 13 retrieves an issue having a high degree of similarity to the genetic test result and the medical information of the patient by using the degree of similarity between word vectors of a keyword included in the genetic test result and the medical information of the patient and a keyword of each issue accumulated in the issue database 221. Further, the issue candidate generation unit 13 generates the issue candidate by the large language model 16 based on an issue retrieval result. The issue candidate generation unit 13 generates the issue candidate related to the genetic test result or the medical information of the patient by the retrieval-augmented generation 22 including the issue database 221.

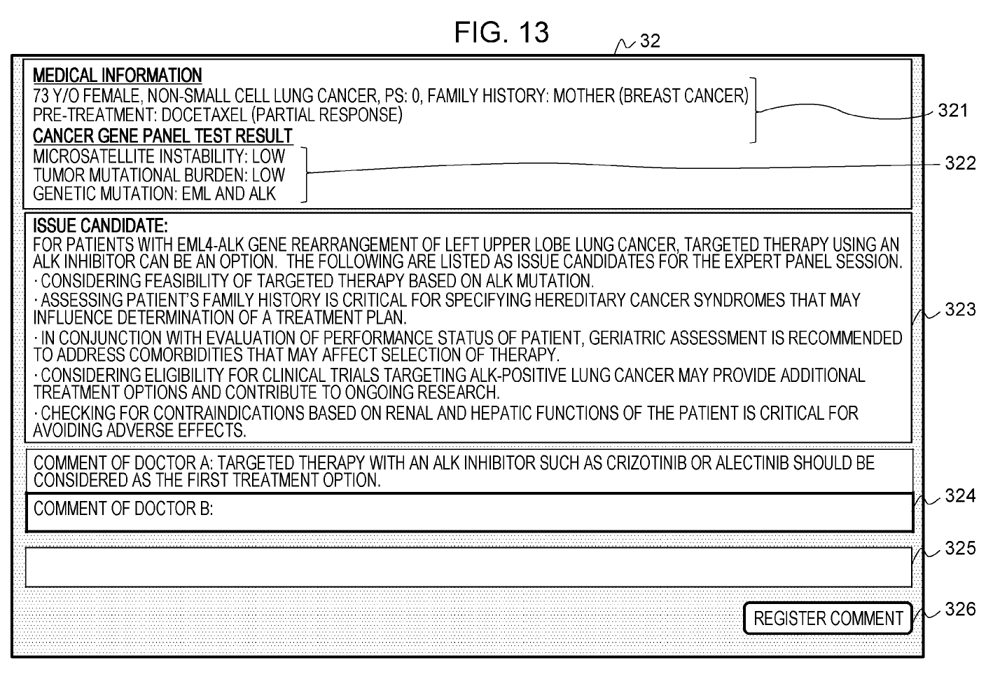

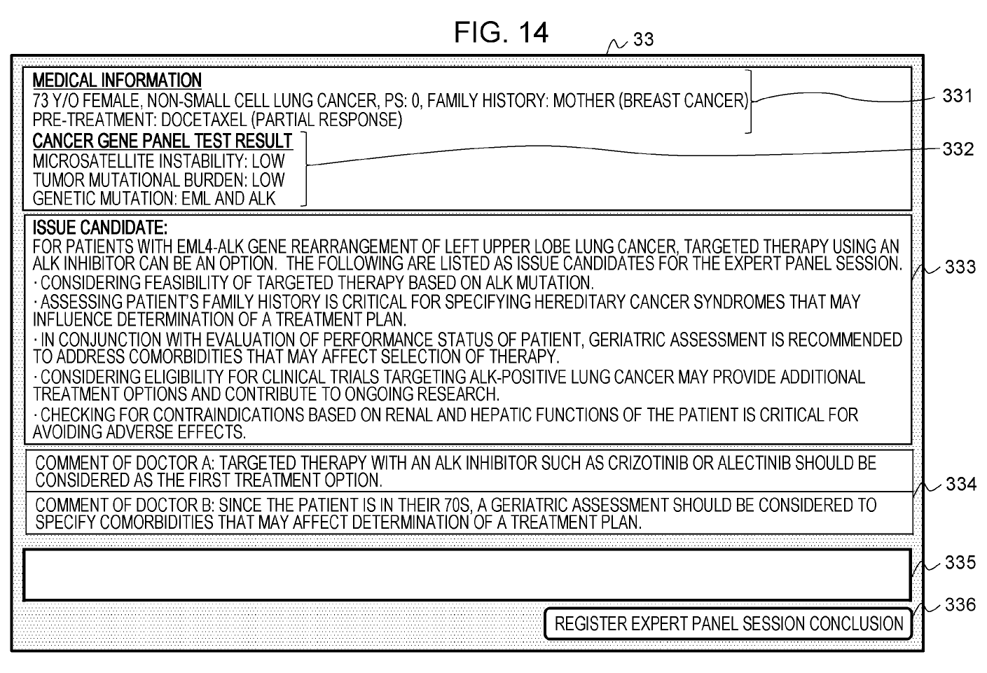

The display control unit 14 displays the genetic test result and the medical information of the patient, and the issue candidate generated by the issue candidate generation unit 13 on the display unit 15. The genetic test result and the medical information of the patient, and the issue candidate generated by the issue candidate generation unit 13 are displayed on the display unit 15. Examples of screens displayed on the display unit 15 are illustrated in FIGS. 13 and 14 described below.

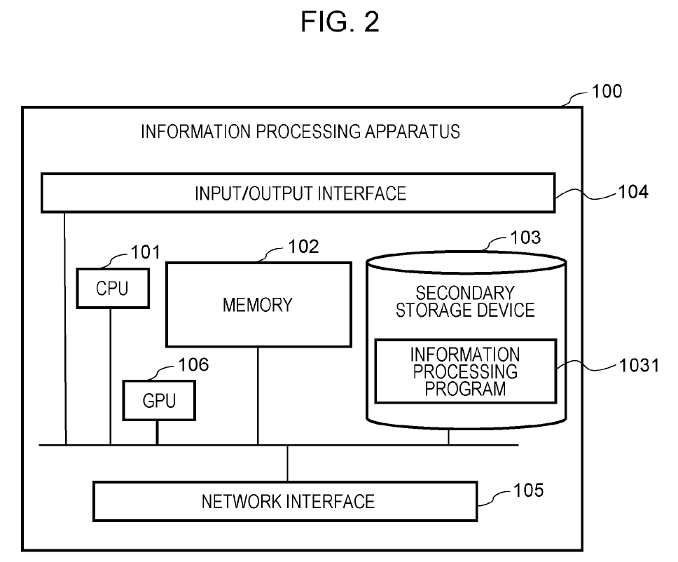

FIG. 2 is a configuration diagram of the information processing apparatus 100.

The information processing apparatus 100 includes a central processing unit (CPU) 101, a memory 102, a secondary storage device 103, an input/output interface 104, a network interface 105, and a graphics processing unit (GPU) 106.

The CPU 101 is one or more central processing units, and integrally controls the information processing apparatus 100. The memory 102 is a temporary storage device, and is, for example, a volatile storage device or a nonvolatile storage device. The secondary storage device 103 is a mass storage device such as a hard disk or a solid state drive (SSD).

The input/output interface 104 is an interface for an input signal from a keyboard or a mouse or an output signal to a display. The network interface 105 is an interface that is connected to a network and exchanges information with other devices. The GPU 106 is a graphics processing unit capable of performing parallel computation.

In the memory 102, each functional module in the information processing apparatus 100 is loaded as a computer program and executed by the CPU 101. Note that processing requiring parallel computation such as a large model may be executed by the GPU 106 as a graphics processing unit.

In addition, an information processing program 1031 is stored in the secondary storage device 103.

The information processing apparatus 100 may be implemented by a virtual machine instead of a physical machine. The information processing apparatus 100 is connected to equipment or an apparatus that controls equipment via the network. The information processing apparatus 100 may be implemented in a cloud environment. In this case, the information processing apparatus 100 is connected to an environment in which equipment is set via the network.

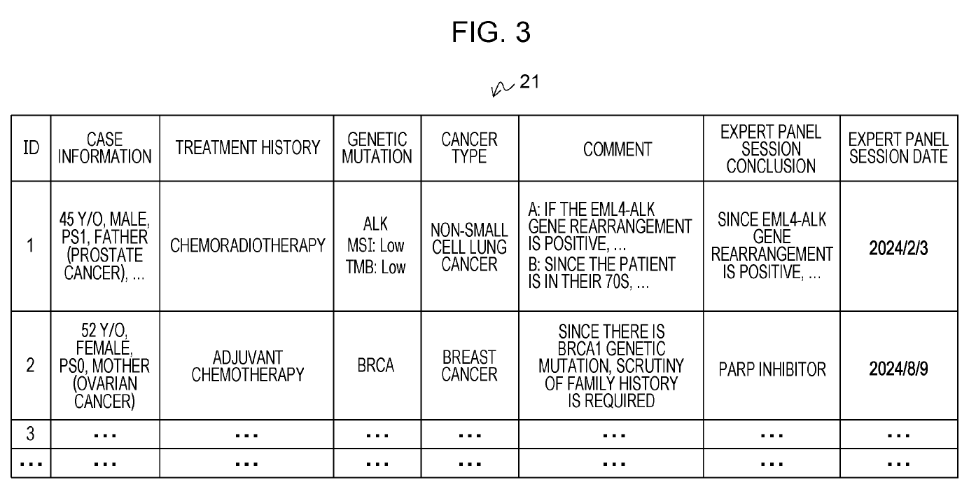

FIG. 3 is a diagram illustrating original past case data.

The past case data is stored in the case database 21.

The past case data includes an ID column, a case information column, a treatment history column, a genetic mutation column, a cancer type column, a comment column, an expert panel session conclusion column, and an expert panel session date column.

In the ID column, an identifier of information stored in a corresponding row is stored.

In the case information column, information regarding a case of a corresponding patient is stored.

In the treatment history column, a treatment history of a corresponding patient is stored.

In the genetic mutation column, information regarding genetic mutation related to a corresponding patient is stored.

In the case information column, age, gender, family history, performance status, and the like as illustrated in FIG. 3 may be mixed, or each item may be divided into different columns or structured.

In the cancer type column, type information of cancer affecting a corresponding patient is stored.

In the comment column, comment information in a natural language format described by a participant of the expert panel session based on the case information and the genetic mutation is stored.

In the expert panel session conclusion column, a conclusion on a corresponding patient in the expert panel session is stored in a natural language format.

In the expert panel session date column, an expert panel session date is stored.

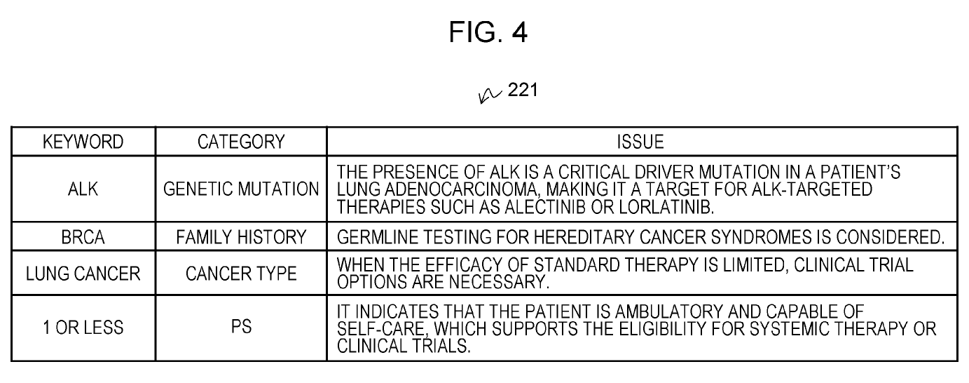

FIG. 4 is a diagram for describing the issue database 221 used in the retrieval-augmented generation 22 for issue generation.

The issue database 221 includes a keyword column, a category column, and an issue column. The issue database 221 can be generated by the large language model classifying the past case data of FIG. 3.

In the keyword column, a keyword related to the issue is stored.

In the category column, category information regarding the issue is stored.

In the issue column, a natural language text regarding the issue is stored.

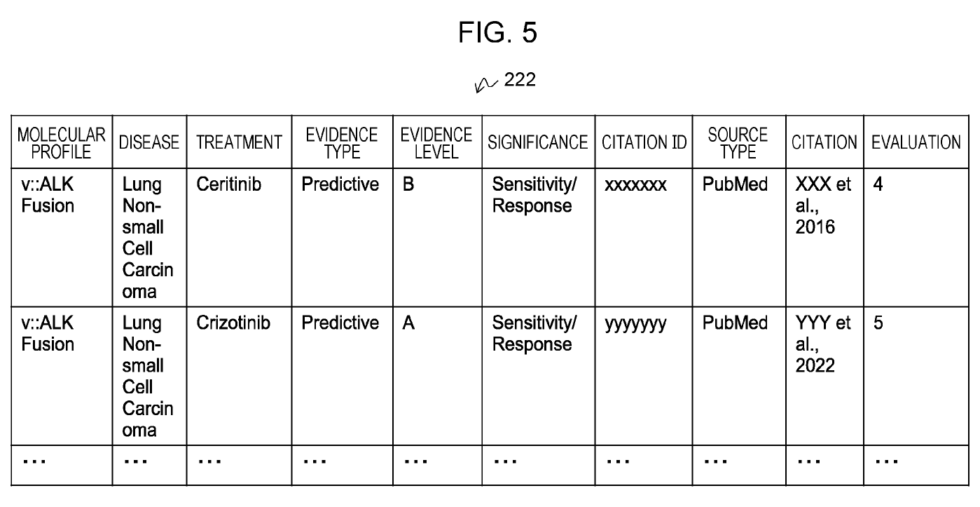

FIG. 5 is a diagram illustrating a medical knowledge database 222 used for the retrieval-augmented generation 22 for issue generation.

The medical knowledge database 222 includes a molecular profile column, a disease column, a treatment column, an evidence column, an evidence level column, a significance column, a citation ID column, a source type column, a citation column, and an evaluation column.

In the molecular profile column, information regarding a target genetic mutation or biomarker is stored. The molecular profile column clarifies a molecular level characteristic for each patient, and thus serves as basic information for personalized medicine and biomarker analysis.

In the disease column, a corresponding disease name, disease category, and the like are stored. Examples of information stored in the disease column include information such as non-small cell lung cancer, breast cancer, and chronic myelogenous leukemia. The disease column may be supplemented with information such as diagnostic criteria and subtypes, for example, stage and grade.

In the treatment column, a therapeutic method and an administered drug applied to the patient are stored. Details such as dose and administration schedules may be stored.

In the evidence column, evidence indicating a therapeutic effect and a correlation is stored. A clinical trial name, a main outcome, and the like may be stored.

In the evidence level column, quality and reliability of data as a basis are ranked and stored. Since the evidence level column can be classified according to strictness and scale of a scientific basis, reliability of judgment can be ensured.

In the significance column, efficacy against corresponding genetic mutation and the presence or absence of a statistical significance are stored. The efficacy is information indicating drug responsiveness, drug resistance, or the like. In the case of representing clinical significance, a P-value, a confidence interval, a hazard ratio, an odds ratio, or the like may also be written together.

In the citation ID column, an internal ID and an index key for specifying a source of data are stored. By associating the citation ID column with the citation column, it is possible to quickly refer to a large amount of data.

In the source type column, information for classifying a type of a citation source is stored. In the source type column, for example, information such as academic papers, guidelines, journal abstracts, databases, and case reports is stored. It is possible to grasp a form and credibility of a source of evidence based on the information in the source type column.

A full reference is stored in the citation column. For example, an author name, a magazine name, a year, a volume (issue), and a page are stored. The information in the source type column facilitates access to the original.

In the evaluation column, scores obtained by comprehensively evaluating quality of the entire evidence and a clinical recommendation grade are stored.

It is possible to transparently and efficiently perform a decision-making process for molecular target therapy and evidence-based medicine based on the information of the medical knowledge database 222.

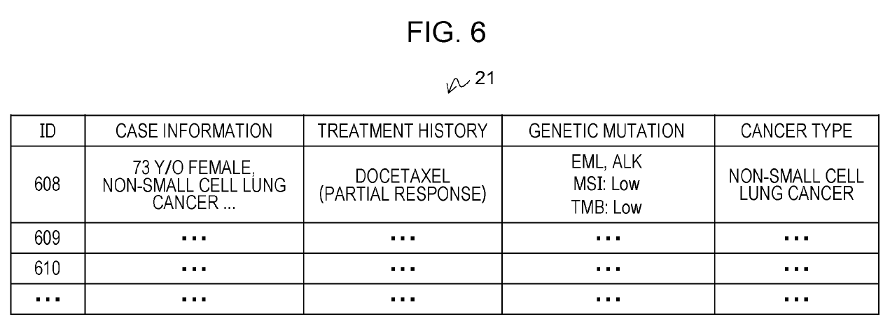

FIG. 6 is a diagram for describing case data of a target patient who is a support target.

The case data is stored in the case database 21. Here, in data regarding a certain patient in the first row, 608 is stored in the ID column, “73 y/o female, non-small cell lung cancer ···” is stored in the case information column, and “docetaxel (partial response)” is stored in the treatment history column. “EML, ALK MSI: Low TMB: Low” is stored in the genetic mutation column of the patient, and “non-small cell lung cancer” is stored in the cancer type column. In the case information column, age, gender, diagnosis name, and the like as illustrated in FIG. 6 may be mixed, or each item may be divided into different columns or structured.

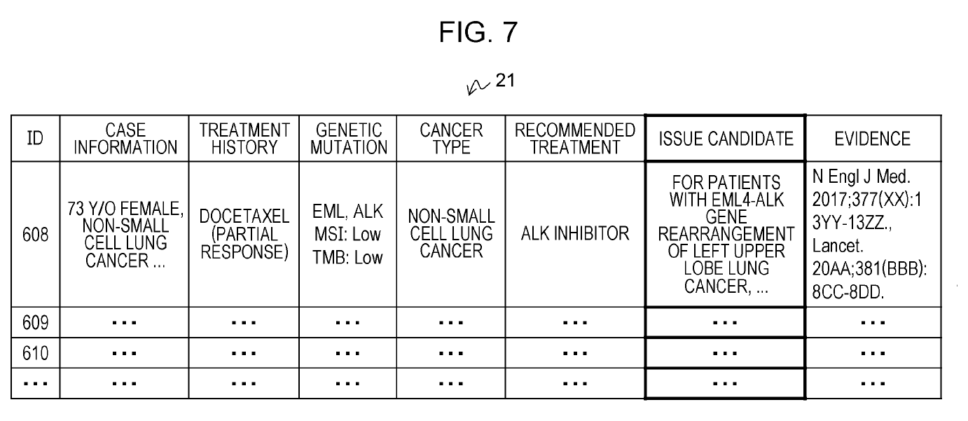

FIG. 7 is a diagram illustrating the issue candidate and the evidence generated by the large language model 16, which is an issue generation AI with the case data of the target patient as an input.

The issue generated by the issue generation AI is stored in the issue column of the case database 21. In data regarding the patient in the first row, “ALK inhibitor” is stored in a recommended treatment column, and “for patients with EML4-ALK gene rearrangement of left upper lobe lung cancer, ···" is stored in an issue candidate column. Furthermore, in the data regarding the patient in the first row, “N Engl J Med. 2017; 377 (XX): 13YY-13ZZ., Lancet. 20AA; 381 (BBB): 8CC-8DD.” is stored in the evidence column.

The large language model 16 uses the retrieval-augmented generation 22 to generate the issue candidate and the evidence related to the issue candidate by using the case data of the target patient as an input. As a result, the participant of the expert panel session can confirm not only raw case information and genetic mutation but also the issue candidate generated based on the case information and the genetic mutation, and input an issue determined by the participant while confirming the evidence related to the issue candidate.

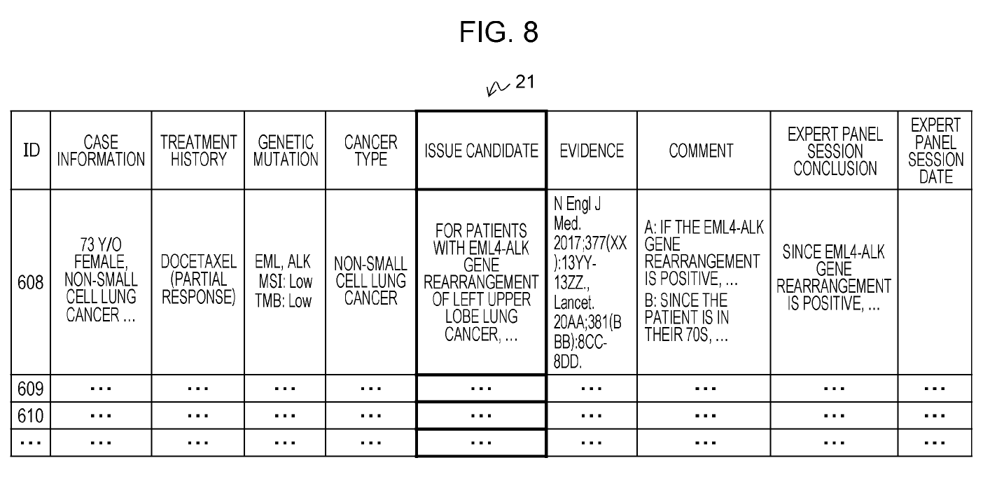

FIG. 8 is a diagram for describing the case database 21.

The case database 21 includes the issue candidate column and the evidence column in addition to the ID column, the case information column, the treatment history column, the genetic mutation column, the cancer type column, the comment column, the expert panel session conclusion column, and the expert panel session date column illustrated in FIG. 3.

The participant of the expert panel session can appropriately write a new comment based on the issue in a natural language format stored in the issue candidate column. Then, in the case database 21, the issue extraction unit 12 extracts a new issue by background processing and updates the issue database 221. As a result, it becomes possible to enrich contents of the issue database 221.

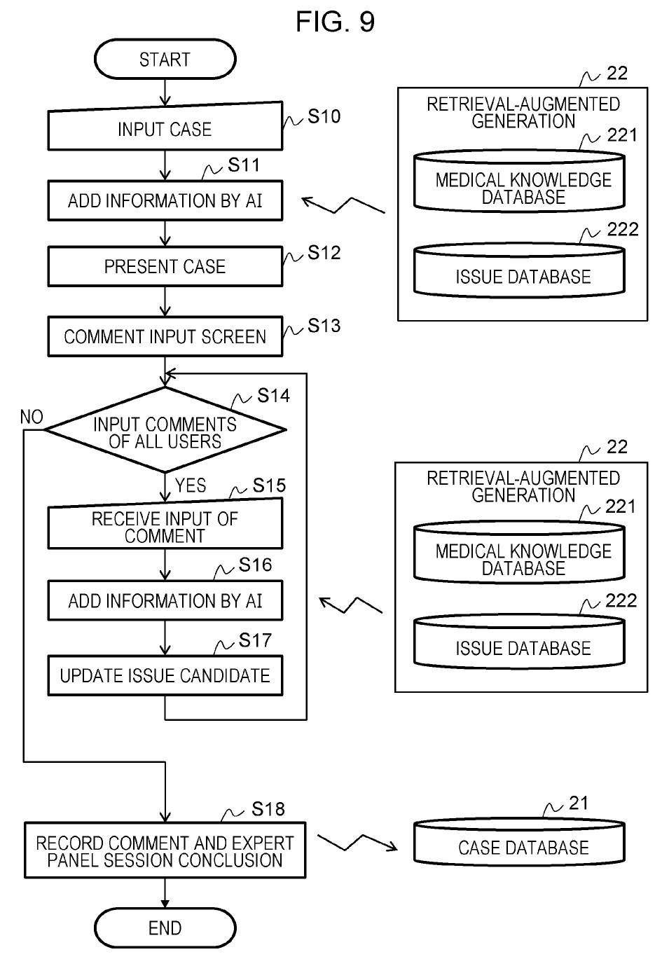

FIG. 9 is a flowchart of preliminary preparation processing for the expert panel session.

Before the expert panel session, the user inputs the case information for the patient. The input unit 11 receives the input of the case information (step S10). The user is, for example, a participant of the expert panel session. The input unit 11 stores the case information in the case database 21. The case information includes age, gender, disease name, and treatment history.

Next, the issue candidate generation unit 13 adds information regarding the issue by the large language model 16 which is the issue generation AI (step S11). The issue candidate generation unit 13 extracts an issue related to a genetic test result or medical information of a patient different from the corresponding patient from the issue database 221 by the large language model 16, and generates an issue candidate for the expert panel session for the corresponding patient based on the extracted issue. The large language model 16 generates the information regarding the issue by using the retrieval-augmented generation 22.

The large language model 16 generates the information regarding the issue by using the retrieval-augmented generation 22. The retrieval-augmented generation 22 includes the issue database 221 and the medical knowledge database 222.

The display control unit 14 presents a case input by the user on the display unit 15 (step S12), and displays a screen including a comment input field on the display unit 15 (step S13).

Then, the input unit 11 determines whether or not inputs of comments of all the users have been received (step S14). When the inputs of all the comments have been received (Yes), the processing proceeds to step S18. When an input of a comment of any participant has not been received (No), the processing proceeds to step S15. A range of all the participants may be changed for each case. In addition, the processing may proceed to step S18 after inputs of a predetermined number (for example, three out of five) of comment input candidates are input, and the processing is not limited.

In step S15, the user inputs the comment with reference to the medical information and the genetic mutation of the patient, and the input unit 11 receives the input of the comment.

Next, the issue candidate generation unit 13 adds information regarding a new issue by the large language model 16 which is the issue generation AI (step S16). The issue candidate generation unit 13 extracts an issue related to a genetic test result or medical information of a patient different from the corresponding patient related to the comment from the issue database 221, and generates an issue candidate for the expert panel session for the corresponding patient based on the extracted issue. The large language model 16 generates the information regarding the issue by using the retrieval-augmented generation 22. The retrieval-augmented generation 22 includes the issue database 221 and the medical knowledge database 222. When the issue extraction unit 12 updates the issue candidate according to the generated issue (step S17), the processing returns to step S14. By updating the issue candidate, the participant of the expert panel session can consider a more accurate comment with reference to the issue candidate reflecting a comment input immediately before by another participant.

In step S18, when the input comment is recorded in the case database 21, the input unit 11 ends the processing of FIG. 9.

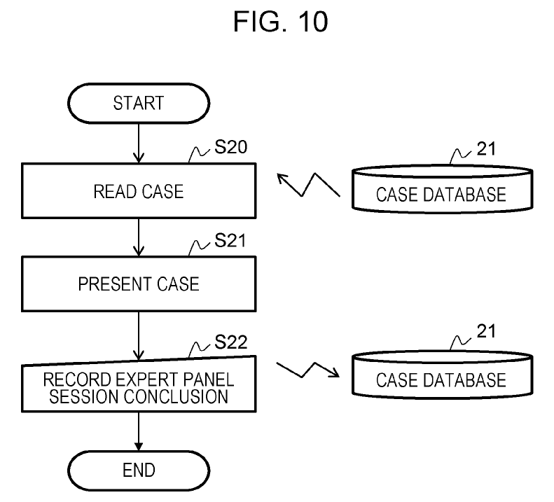

FIG. 10 is a flowchart of in-session processing of the expert panel session.

First, a case of a certain patient is read from the case database 21 (step S20), and the display control unit 14 presents the case of the patient on the display unit 15 (step S21). Then, when an input of a conclusion of the expert panel session is received, the input unit 11 records the conclusion in the case database 21 (step S22), and ends the processing of FIG. 10.

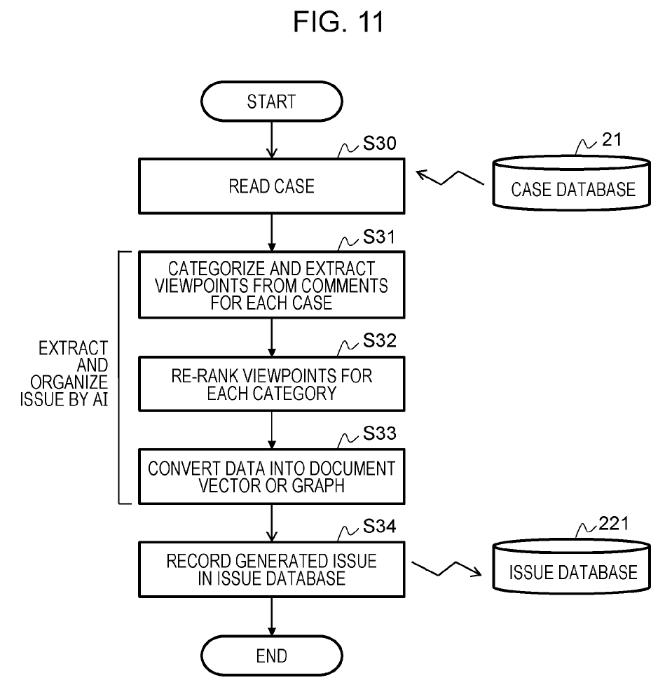

FIG. 11 is a flowchart of the background processing.

First, the input unit 11 reads a case of a certain patient from the case database 21 (step S30). Then, when viewpoints are categorized and extracted from comments for each case by the large language model 16 which is the issue generation AI (step S31), the issue extraction unit 12 re-ranks the viewpoints for each category (step S32). Then, the issue extraction unit 12 converts issue data into a document vector or a graph (step S33). The processing in steps S31 to S33 is processing of extracting and organizing the issue by the large language model 16.

Then, when the generated issue is recorded in the issue database 221 (step S34), the issue extraction unit 12 ends the processing of FIG. 11.

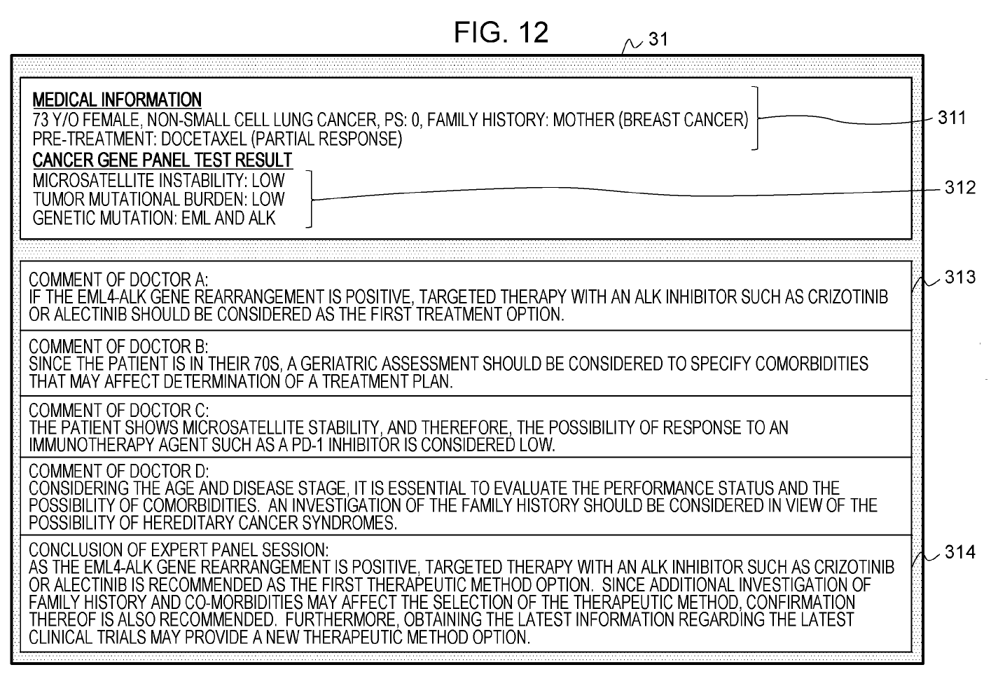

FIG. 12 illustrates a comment input screen 31 according to a related art.

The comment input screen 31 includes medical information 311, a cancer gene panel test result 312, a comment text box 313, and an expert panel session conclusion text box 314.

The medical information 311 indicates “73 y/o female, non-small cell lung cancer, PS: 0, family history: mother (breast cancer), pre-treatment: docetaxel (partial response)”. As the medical information 311, contents of the case information column, contents of the treatment history column, and contents of a cancer history column of the case database 21 are displayed.

The cancer gene panel test result 312 describes the following.
“Microsatellite Instability: Low
Tumor Mutational Burden:
Low Genetic Mutation: EML and ALK”

As the cancer gene panel test result 312, contents of a cancer gene panel test result column (not illustrated) of the case database 21 are displayed.

In the comment text box 313, comments of doctors A to D are described. As a display content of the comment text box 313, contents of the comment column of the case database 21 are displayed.

In the expert panel session conclusion text box 314, the conclusion of the expert panel session is described in a natural language format. The conclusion of the expert panel session is described in the expert panel session conclusion text box 314 with reference to the medical information 311, the cancer gene panel test result 312, and the comment text box 313.

FIG. 13 illustrates a comment input screen 32 of the present embodiment.

The comment input screen 32 includes medical information 321, a cancer gene panel test result 322, an issue candidate text box 323, a comment text box 324, an expert panel session conclusion text box 325, and a comment registration button 326. Information regarding a predetermined patient is displayed on the comment input screen 32, and an input field for inputting a comment regarding the patient is provided.

As the medical information 321, information similar to the medical information 311 of FIG. 12 is displayed.

As the cancer gene panel test result 322, information similar to that of the cancer gene panel test result 312 in FIG. 12 is displayed.

In the issue candidate text box 323, a candidate for an issue to be described as a comment on the patient is described in a natural language format. Here, the following is described.

“For patients with EML4-ALK gene rearrangement of left upper lobe lung cancer, targeted therapy using an ALK inhibitor can be an option. The following are listed as issue candidates for the expert panel session.
Considering feasibility of targeted therapy based on ALK mutation.
Assessing a patient's family history is critical for specifying hereditary cancer syndromes that may influence determination of a treatment plan.
In conjunction with evaluation of a performance status of the patient, a geriatric assessment is recommended to address comorbidities that may affect selection of a therapeutic method.
Considering eligibility for clinical trials targeting ALK-positive lung cancer may provide additional treatment options and contribute to ongoing research.
Checking for contraindications based on renal and hepatic functions of the patient is critical for avoiding adverse effects.”

The information in the issue candidate text box 323 is generated using the retrieval-augmented generation 22 when the medical information and genetic mutation of the patient are input into the large language model 16. By referring to the issue candidate, each doctor can appropriately write a comment.

In the comment text box 324, the comment of the doctor A is described, and a comment field for the doctor B is displayed in an input-enabled state.

The expert panel session conclusion text box 325 is empty and is displayed in a disabled state.

The comment registration button 326 is a button for registering the input comment. By clicking the comment registration button 326, the newly input comment is registered in the comment column of the case database 21. Then, the large language model 16 generates a new issue candidate including the new comment. Then, the display control unit 14 updates the issue candidate text box 323 with the new issue candidate.

FIG. 14 illustrates a comment input screen 33 of the present embodiment.

The comment input screen 33 includes medical information 331, a cancer gene panel test result 332, an issue candidate text box 333, a comment text box 334, an expert panel session conclusion text box 335, and an expert panel session conclusion registration button 336.

As the medical information 331, information similar to the medical information 311 of FIG. 12 is displayed.

As the cancer gene panel test result 332, information similar to the cancer gene panel test result 312 of FIG. 12 is displayed.

In the issue candidate text box 333, information similar to that in the issue candidate text box 323 of FIG. 13 is displayed.

In the comment text box 334, the comment of the doctor A in FIG. 13 is displayed, and the comment of the doctor B is displayed.

The expert panel session conclusion text box 335 is displayed in a state in which a text can be input. The expert panel session conclusion registration button 336 is a button for registering the input expert panel session conclusion. By clicking the expert panel session conclusion registration button 336, the newly input information on the conclusion of the expert panel session is registered in the case database 21.

Hereinafter, the configuration and effect of the present invention will be described.

An information processing system for an expert panel session, the information processing system including:
an input unit (11) that receives inputs of a genetic test result and medical information of a patient who has undergone a genetic test, and a comment regarding clinical interpretation or treatment proposal for the genetic test result or the medical information of the patient from a participant of the expert panel session, and accumulates the inputs in a case database (21);
an issue extraction unit (12) that extracts an issue obtained by classifying and summarizing a plurality of the comments on the genetic test result and the medical information in the case database (21) by a large language model (16) and accumulates the extracted issue in an issue database (221);
an issue candidate generation unit (13) that extracts an issue related to a genetic test result or medical information of a patient different from the patient from the issue database (221) by the large language model (16), and generates an issue candidate for the expert panel session for the patient based on the extracted issue; and
a display control unit (14) that displays the genetic test result and the medical information of the patient, and the issue candidate on a display unit (15).

As a result, it is possible to suitably support work of experts participating in the expert panel session.

The information processing system according to [1], in which the medical information includes age, gender, disease name, case information, and treatment history.

As a result, it is possible to generate the issue candidate according to the age, gender, disease name, case information, and treatment history.

The information processing system according to [1], in which when the issue is created by classifying and summarizing the plurality of comments on the genetic test results and the medical information in the case database (21) by the large language model (16), the issue extraction unit (12) records the issue in the issue database (221) by dividing the issue into a plurality of elements including a keyword, a category, and a consideration item.

As a result, the large language model can appropriately refer to the issue database when generating the issue candidate.

The information processing system according to [3], in which the issue extraction unit (12) further extracts an issue having a high degree of similarity by using the degree of similarity between word vectors of a keyword included in the genetic test result or the medical information and a keyword of each issue accumulated in the issue database (221) when extracting the issue obtained by classifying and summarizing the comments on the genetic test result and the medical information of the patient.

As a result, the issue database can be rearranged in advance in descending order of the degree of similarity, and the issue candidate can be appropriately extracted.

The information processing system according to [1], in which the issue candidate generation unit (13) generates the issue candidate related to the genetic test result or the medical information of the patient by the large language model (16) and retrieval-augmented generation (22).

As a result, the large language model can appropriately generate the issue candidate by using the retrieval-augmented generation in which the issue is organized in advance.

The information processing system according to [1], in which the issue candidate generation unit (13) further generates evidence related to the issue candidate in addition to the issue candidate related to the genetic test result or the medical information of the patient by the large language model (16) and the retrieval-augmented generation (22).

As a result, the participant of the expert panel session can evaluate validity of the issue candidate by referring to the evidence.

The information processing system according to [5], in which the issue candidate generation unit (13) generates the issue candidate related to the genetic test result or the medical information of the patient by the large language model (16) and the retrieval-augmented generation (22) with reference to the comments.

As a result, the large language model can appropriately regenerate the issue candidate based on a newly input comment.

The information processing system according to [5], in which the retrieval-augmented generation (22) includes an issue database (221) and a medical knowledge database (222).

As a result, the issue candidate generation unit can appropriately generate the issue based on the issue and the medical knowledge.

The information processing system according to [1], in which the issue candidate generation unit (13) generates the issue candidate related to the genetic test result or the medical information of the patient by the large language model (16) and a knowledge graph.

Therefore, the issue candidate can mechanically be generated based on the knowledge graph.

The information processing system according to [1], in which

the input unit (11) receives a treatment result of the patient as an input, and

the issue candidate generation unit (13) adds, to the issue candidate, prognosis information of a case in which the same genetic mutation as genetic mutation of the patient is detected, or a case in which a treatment drug corresponding to the genetic mutation of the patient is prescribed.

As a result, the issue candidate generation unit can generate an appropriate issue candidate that reflects a treatment result on the patient.

The information processing system according to [1], in which when the input unit (11) receives an input of a new comment regarding clinical interpretation or treatment proposal for the genetic test result or the medical information of the patient from the participant of the expert panel session, the issue extraction unit (12) updates the issue accumulated in the issue database (221).

As a result, it is possible to store a new issue reflecting a new comment in the issue database together with an input of the new comment.

An information processing method for an expert panel session, the information processing method including:
receiving, by an input unit (11), inputs of a genetic test result and medical information of a patient who has undergone a genetic test, and a comment regarding clinical interpretation or treatment proposal for the genetic test result or the medical information of the patient from a participant of the expert panel session, and accumulating the inputs in a case database (21);
extracting, by an issue extraction unit (12), an issue obtained by classifying and summarizing a plurality of the comments on the genetic test result and the medical information in the case database (21) by a large language model (16) and accumulating the extracted issue in an issue database (221);
extracting, by an issue candidate generation unit (13), an issue related to a genetic test result or medical information of a patient different from the patient from the issue database (221) by the large language model (16);
generating, by the issue candidate generation unit (13), an issue candidate for the expert panel session for the patient based on the extracted issue; and
displaying, by a display control unit (14), the genetic test result and the medical information of the patient, and the issue candidate on a display unit (15).

As a result, it is possible to suitably support work of experts participating in the expert panel session.

The information processing method according to [12], further including:
receiving, by the input unit (11), an input of a new comment regarding clinical interpretation or treatment proposal for the genetic test result or the medical information of the patient;
extracting, by the issue candidate generation unit (13), a new issue from the issue database (221) with reference to the new comment;
generating, by the issue candidate generation unit (13) based on the extracted new issue, a new issue candidate for the expert panel session for the patient; and
displaying, by the display control unit (14), the genetic test result and the medical information of the patient, and the new issue candidate on the display unit (15).

As a result, it is possible to update the issue candidate each time the expert participating in the expert panel session inputs a comment and to suitably support the next expert to input a comment.

### Modified Example

The present invention is not limited to the embodiments described above, but includes various modified examples. For example, the above-described embodiments have been described in detail in order to explain the present invention in an easy-to-understand manner, and the present invention is not necessarily limited to those having all the configurations described. A part of a configuration of an embodiment can be replaced with a configuration of another embodiment, and a configuration of an embodiment can be added with a configuration of another embodiment. In addition, a part of the configuration of each embodiment can be added with another configuration, can be deleted, and can be replaced with another configuration.

Some or all of the above-described configurations, functions, processing units, processing means, and the like may be implemented by hardware such as an integrated circuit. Each of the above-described configurations, functions, and the like may be implemented by software in a manner in which a processor interprets and executes a program for implementing each function. Information such as a program, a table, and a file for implementing each function can be stored in a recording device such as a memory, a hard disk, and a solid state drive (SSD), or a recording medium such as a flash memory card or a digital versatile disk (DVD).

In each embodiment, the control lines and information lines indicate those that are considered necessary for explanation, and do not necessarily indicate all the control lines and information lines in the product. In practice, it can be considered that almost all configurations are interconnected.

Modified examples of the present invention include the following (a) to (f).
a) The present invention is not limited to support of an expert panel session regarding gene therapy, and may be used for support of an expert panel session for any medical practice.
(b) The case database, the issue database, and the medical knowledge database of the present invention do not have to be provided in the information processing apparatus, and may be implemented by accessing databases provided in an external server.
(c) The present invention is not limited to an expert panel session regarding a medical practice, and may be applied to an expert panel session in an arbitrary field.
(d) The present invention is not limited to a single information processing apparatus, and may be implemented by an information processing system in which a plurality of computers operate in conjunction with each other.
(e) When the large language model 16 according to the present invention generates the issue, it is not limited to using the retrieval-augmented generation 22, and the knowledge graph may be used, and there is no limitation. The knowledge graph is a knowledge network in which various types of knowledge are systematically connected and represented by a graph structure, and enables machine processing for unstructured data such as a natural language text and a speech.
(f) After converting the plurality of comments on the genetic test result and the medical information in the case database 21 into the knowledge graph by the large language model 16, the issue extraction unit 12 may extract and accumulate an issue related thereto in the knowledge graph. Then, the issue candidate generation unit may generate the issue candidate with reference to the knowledge graph.

What is claimed is:

1. An information processing system for an expert panel session, the information processing system comprising:
an input unit that receives inputs of a genetic test result and medical information of a patient who has undergone a genetic test, and a comment regarding clinical interpretation or treatment proposal for the genetic test result or the medical information of the patient from a participant of the expert panel session, and accumulates the inputs in a case database;
an issue extraction unit that extracts an issue obtained by classifying and summarizing a plurality of the comments on the genetic test result and the medical information in the case database by a large language model and accumulates the extracted issue in an issue database;
an issue candidate generation unit that extracts an issue related to a genetic test result or medical information of a patient different from the patient from the issue database by the large language model, and generates an issue candidate for the expert panel session for the patient based on the extracted issue; and
a display control unit that displays the genetic test result and the medical information of the patient, and the issue candidate on a display unit.

2. The information processing system according to claim 1, wherein the medical information includes age, gender, disease name, case information, and treatment history.

3. The information processing system according to claim 1, wherein when the issue is created by classifying and summarizing the plurality of comments on the genetic test results and the medical information in the case database by the large language model, the issue extraction unit records the issue in the issue database by dividing the issue into a plurality of elements including a keyword, a category, and a consideration item.

4. The information processing system according to claim 3, wherein the issue extraction unit further extracts an issue having a high degree of similarity by using the degree of similarity between word vectors of a keyword included in the genetic test result or the medical information and a keyword of each issue accumulated in the issue database when extracting the issue obtained by classifying and summarizing the comments on the genetic test result and the medical information of the patient.

5. The information processing system according to claim 1, wherein the issue candidate generation unit generates the issue candidate related to the genetic test result or the medical information of the patient by the large language model using retrieval-augmented generation.

6. The information processing system according to claim 1, wherein the issue candidate generation unit further generates evidence related to the issue candidate in addition to the issue candidate related to the genetic test result or the medical information of the patient by the large language model using retrieval-augmented generation.

7. The information processing system according to claim 5, wherein the issue candidate generation unit generates the issue candidate related to the genetic test result or the medical information of the patient by the large language model using the retrieval-augmented generation with reference to the comments.

8. The information processing system according to claim 5, wherein the retrieval-augmented generation includes an issue database and a medical knowledge database.

9. The information processing system according to claim 1, wherein the issue candidate generation unit generates the issue candidate related to the genetic test result or the medical information of the patient by the large language model and a knowledge graph.

10. The information processing system according to claim 1, wherein
the input unit receives a treatment result of the patient as an input, and
the issue candidate generation unit adds, to the issue candidate, prognosis information of a case in which the same genetic mutation as genetic mutation of the patient is detected, or a case in which a treatment drug corresponding to the genetic mutation of the patient is prescribed.

11. The information processing system according to claim 1, wherein when the input unit receives an input of a new comment regarding clinical interpretation or treatment proposal for the genetic test result or the medical information of the patient from the participant of the expert panel session, the issue extraction unit updates the issue accumulated in the issue database.

12. An information processing method for an expert panel session, the information processing method comprising:
receiving, by an input unit, inputs of a genetic test result and medical information of a patient who has undergone a genetic test, and a comment regarding clinical interpretation or treatment proposal for the genetic test result or the medical information of the patient from a participant of the expert panel session, and accumulating the inputs in a case database;
extracting, by an issue extraction unit, an issue obtained by classifying and summarizing a plurality of the comments on the genetic test result and the medical information in the case database by a large language model and accumulating the extracted issue in an issue database;
extracting, by an issue candidate generation unit, an issue related to a genetic test result or medical information of a patient different from the patient from the issue database by the large language model;
generating, by the issue candidate generation unit, an issue candidate for the expert panel session for the patient based on the extracted issue; and
displaying, by a display control unit, the genetic test result and the medical information of the patient, and the issue candidate on a display unit.

13. The information processing method according to claim 12, further comprising:
receiving, by the input unit, an input of a new comment regarding clinical interpretation or treatment proposal for the genetic test result or the medical information of the patient;
extracting, by the issue candidate generation unit, a new issue from the issue database with reference to the new comment;
generating, by the issue candidate generation unit based on the extracted new issue, a new issue candidate for the expert panel session for the patient; and
displaying, by the display control unit, the genetic test result and the medical information of the patient, and the new issue candidate on the display unit.

14. The information processing system according to claim 1, wherein the expert panel includes a molecular tumor board (MTB)
